SEQUENCE LISTING (3 sequences)

SEQ ID NO: 1 - protein, 101 aa, Human
MLQPLQESGIIMEQALRKNRLQLGTEQPGCTPDASGTWCLLWRMGQLPHCPGARASDPGAKVCLFHFWELAVFARLSGPQASHCPPGITFLQDHGEDDMRC

SEQ ID NO: 2 - protein, 169 aa, Human
MQCLLPYQSKEPSCLPPLPLNLPLPPCLCPLLQLNAAMTRKEKTKEGQRAAQFSAGADAGSGGGLSRQKDTKRPMLLVIHDVVLELLTSSDCHANPRKYPTCQKSEVLGVSIYVSICPSTRPRDKNKTKKRCQVLEAVLVSKPSGSCHQGSFEIVPHVKGNLAFTSSNH

SEQ ID NO: 3 - protein, 42 aa, Human
MFHQILVGLKKHSSFIPLRIYEIRRYWSSAVCPASGIVQSRC

(19) United States
(12) Patent Application Publication
(10) Pub. No.: US 2022/0291218 A1
(43) Pub. Date: Sep. 15, 2022

(54) METHOD OF IMPROVING EFFICACY OF MELANOMA TREATMENT

(71) Applicant: New York University, New York, NY (US)

(72) Inventors: Iman Osman, Jersey City, NJ (US); Keith Giles, New York, NY (US)

(21) Appl. No.: 17/705,960

(22) Filed: Mar. 28, 2022

Related U.S. Application Data
(62) Division of application No. 16/413,418, filed on May 15, 2019.
(60) Provisional application No. 62/671,511, filed on May 15, 2018.

(51) Int. Cl. G01N 33/574; C07K 16/28; A61K 39/395; G01N 33/564; A61P 35/00

(52) U.S. Cl. CPC G01N 33/5743; C07K 16/2818; C07K 16/2878; A61K 39/39558; G01N 33/564; A61P 35/00; A61K 2039/505

(57) ABSTRACT

Provided are compositions and methods for determining whether or not an individual who has cancer is likely to develop immune-related adverse events (irAEs) because of treatment with an immune checkpoint inhibitor such as an anti-Programmed cell death protein 1 (anti-PD-1) checkpoint inhibitor, and/or anti-Cytotoxic T-lymphocyte-associated protein 4 (anti-CTLA-4) checkpoint inhibitor. Also provided are methods for treating an individual who has cancer with a checkpoint inhibitor and an agent to reduce the risk of toxicity from the checkpoint inhibitor, as well as administering agents to prevent or reduce the risk of toxicity during treatment.

### CROSS REFERENCE TO RELATED APPLICATIONS

This application is a divisional of U.S. patent application Ser. No. 16/413,418, filed on May 15, 2019, which claims priority to U.S. provisional patent application No. 62/671,511, filed May 15, 2018, the entire disclosure of each of which are incorporated herein by reference.

### SEQUENCE LISTING

The instant application contains a Sequence Listing which has been submitted electronically in ASCII format and is hereby incorporated by reference in its entirety. Said ASCII file, created on May 24, 2019, is named Giles_NYU.TXT, and is 3,083 bytes in size.

### BACKGROUND

Immune checkpoint inhibitors (ICI) target cytotoxic T lymphocyte-associated antigen 4 (CTLA-4, e.g. ipilimumab) or programmed cell death protein 1 (PD-1, e.g. nivolumab, pembrolizumab) to promote T-cell mediated anti-tumor immunity and produce durable clinical benefit in a subset of patients with advanced melanoma [1]. More recently, the combination of anti-CTLA-4 and anti-PD-1 has been shown to be more efficacious than single agent therapy [2]. Despite this progress a substantial proportion of patients receiving ICI develop immune-related adverse events (irAEs) [3], which are often more severe in patients receiving combination regimens [4]. IrAEs can necessitate systemic immunosuppression therapy and/or treatment termination [5]. Hence, there is an urgent clinical need to identify patients who are more likely to develop severe irAEs, particularly as more patients receive these immune therapies due to the approval of ICI for other cancer types (e.g. bladder, lung), and in the adjuvant setting for stage III/IV melanoma [6,7]. A biomarker predictive of immunotherapy toxicity would facilitate a personalized approach to patient management, enabling more-effective combination treatments to be used in patients who are less likely to develop severe irAEs. Additionally, identifying toxicity-prone patients would improve the clinical management of irAEs by allowing for earlier or prophylactic interventions to mitigate toxicities.

Although there is intense interest in identifying markers that predict the efficacy of ICIs [8,9], pre-treatment biomarkers of ICI toxicity and irAEs have been less thoroughly investigated. Changes in IL-17, CD8 T-cell clonal expansion, eosinophil counts, and markers of neutrophil activation have been associated with specific irAEs after treatment induction, but did not predict toxicity development when tested at baseline [10-12]. Several other potential baseline risk factors for development of irAEs from ICI have been suggested, including a family history of autoimmune diseases, previous viral infections, and use of medicines with known autoimmune toxicities [13,14], but these require further validation. More recently, in a small study, the baseline microbiome composition of melanoma patients was found to be associated with onset of immune mediated colitis following anti-CTLA-4 treatment [15]; while this finding demonstrates the potential utility of pre-treatment/baseline biomarkers of toxicity development, it does not reflect the spectrum of different irAEs associated with ICI. Thus, there is an ongoing and unmet needs for new approaches to identifying individuals who are susceptible to developing irAEs, and for treating such patients with this information in hand. The present disclosure is pertinent to these needs.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Validation of a proteomic microarray for measurement of serum antibodies. (A) Intra-chip reproducibility was assessed by comparing probe intensity readings for duplicate spots from 10 independent serum samples/chips. Linear regression analysis was used to determination the correlation between spots within chips. To assess interchip reproducibility, probe intensity readings were assessed in the same 10 serum samples across two distinct microarrays on separate occasions, and linear regression analysis was used to determine the correlation between chips. (B) Comparison of probe array signal intensities for anti-CTLA-4 antibodies from serum samples (n=39) from melanoma patients taken before and after anti-CTLA-4 ICI treatment. Top, raw array scans of duplicate anti-CTLA-4 spots for pre- and post-anti-CTLA-4 samples from patient 10-262. Bottom, graph showing combined anti-CTLA-4 array signals (mean±sd) for all pre- and post-treatment samples. *, p<0.0001.

FIG. 2. Antibodies from baseline sera of melanoma patients are associated with ICI toxicity. (A) Volcano plot of differential antibody levels from baseline sera comparing none/mild vs. severe toxicity for anti-CTLA-4-treated patients (n=37). Filtered antibodies are highlighted in blue, and curated antibodies are indicated in red (downregulated with severe toxicity) or purple (upregulated with severe toxicity). (B) As for (A), but comparing no/mild vs. severe toxicity for anti-PD-1-treated patients (n=27). (C) As for (A), but comparing mild vs. severe toxicity for anti-CTLA-4 and anti-PD-1 combination treated patients (n=11). (D) Boxplots showing probe intensities for the 15 most differentially expressed antibodies (DE; based on p-values) between sera from antiCTLA-4 patients (n=37) with no/mild toxicity (blue) vs. those with severe toxicity (orange). Data represent median probe intensities ±sd. (E) As for (D), but for samples comparing no/mild vs. severe toxicity for anti-PD-1-treated patients (n=27). (F) As for (D), but for samples comparing mild vs. severe toxicity for combination anti-CTLA-4 and anti-PD-1-treated patients (n=11).

FIG. 3. Functional significance of toxicity-associated antibodies. (A) Functional pathway enrichment (WikiPathways) of protein targets from the filtered set of toxicity-associated antibodies from anti-CTLA-4-treated patients. (B) As for (A), but for anti-PD-1-treated patients. (C) As for (A), but for combination-treated patients. (D) Summary of immune toxicity associations for protein targets of top 15 DE termination-associated antibodies from anti-CTLA-4-treated patients. (E) As for (D), but for anti-PD-1-treated patients. (F) As for (D), but for combination-treated patients.

FIG. 4. Development of classification models to predict immunotherapy toxicity using antibodies from pre-treatment melanoma patient sera. (A) Scatterplot showing distribution of decision values from support vector machine (SVM) classifier models based on “filtered” antibody (feature) lists for prediction of severe toxicity. Data summarizes training and testing results from 100 repetitions of 5 fold cross validation for pre-anti-CTLA-4 samples. Gold circles represent true positives (severe toxicity sample called as severe toxicity) and green crosses represent true negatives (no/mild toxicity sample called as no/mild toxicity). Red circles represent false negatives (severe toxicity sample called as no/mild toxicity) and blue crosses represent false positives (no/mild toxicity called as severe toxicity). (B) As for (A), but summarizing 100 repetitions of 5-fold cross validation for anti-PD-1 samples. (C) As for (A), but summarizing 100 repetitions of 3-fold cross validation for anti-CTLA-4 and anti-PD-1 combination samples. (D) Summary of accuracy, sensitivity, and specificity cross validation statistics based on SVM models for prediction of toxicity in anti-CTLA-4 samples (no/mild toxicity, n=30; severe, n=9). (E) As for (D), but for anti-PD-1 samples (no/mild toxicity, n=19; severe, n=9). (F) As for D, but for combined anti-CTLA-4 and anti-PD-1 samples (mild toxicity, n=4; severe, n=7).

FIG. 5. Pre- vs. post-anti-CTLA-4 treatment reproducibility (n=39). (A) Correlation plot of global antibody profiles (array probe intensities) for pre- and post-anti-CTLA-4 treatment samples from patient 09-035. (B) Summary of correlation (r²) values for antibody profiles, including mean and standard deviation, between pre- and post-anti-CTLA-4 treatment samples (n=39 pairs).

FIG. 6. Cross validation ROC curves for anti-PD-1 no/mild vs. severe toxicity using 28 validated autoantibodies show excellent specificity and sensitivity for prediction of toxicity. Left Panel: Using 28 validated autoantibodies and samples from Dataset 1 (Gowen et al., 2018), an SVM model (radial basis kernel, sigma=2\hat{ }-5, C=10) was built and evaluated using 10-fold cross-validation. The associated receiver operating characteristic (ROC) curve using decision values from testing folds is shown. AUC=0.945. Right Panel: As for Left, but instead using samples from Dataset 2 (validation cohort). AUC=0.978.

FIG. 7. Cross validation ROC curves for anti-PD-1+anti-CTLA-4 (combination) no/mild vs. severe toxicity using 26 validated autoantibodies show excellent specificity and sensitivity for prediction of toxicity. Left Panel: Using 26 validated autoantibodies and samples from Dataset 1 (Gowen et al., 2018), an SVM model (radial basis kernel, sigma=2\hat{ }-5, C=10) was built and evaluated using 3-fold cross-validation. The associated receiver operating characteristic (ROC) curve using decision values from testing folds is shown. AUC=0.972. Right Panel: As for Left, but instead using samples from Dataset 2 (validation cohort) and 5-fold cross-validation. AUC=1.000.

### SUMMARY

In this disclosure, antibody levels were analyzed in sera from melanoma patients. The results show significant differences in a subset of IgG antibodies in pre-treatment sera from patients who developed severe irAEs compared to those with no or mild irAEs. The pathway and localization analyses (KEGG, Reactome, UniProt) revealed that the autoantibodies (autoAbs) identified largely targeted intracellular components, such as nuclear and mitochondrial antigens, suggesting a susceptibility to systemic autoimmune toxicity. These data indicate that a subset of melanoma patients has a baseline autoimmune susceptibility that is characterized by a repertoire of specific preexisting autoAbs, which predicts and exacerbates the development of toxicity during immune checkpoint inhibitor therapy. Therefore, detection of baseline autoAbs provides: (i) identification of melanoma patients likely to develop severe irAEs from treatment, which can guide therapy selection or proactively and preemptively manage toxicity, and (ii) provide insight into new approaches to prevent this toxicity without compromising the anti-tumor immune response.

In embodiments, the disclosure provides compositions and methods for determining whether or not an individual who has cancer is likely to develop irAEs because of treatment with an immune checkpoint inhibitor. In embodiments, the disclosure includes testing a biological sample from an individual to determine the presence, absence, and/or amounts of autoAbs by exposing the biological sample to proteins on a protein array, wherein the proteins can be bound with specificity by the autoAbs, if present in the sample. In embodiments, the proteins are any combination of proteins described herein, and include but are not necessarily limited to those proteins described in Table 1.

In embodiments, a composition of matter formed during such a test is provided. For example, in one embodiment, the disclosure provides a composition of matter comprising a biological sample obtained from an individual who has cancer and who was not treated with a checkpoint inhibitor prior to obtaining the biological sample, and a plurality of proteins attached to a substrate, the plurality of proteins being selected from the proteins of Table 1. In embodiments, the plurality of proteins includes least TNFRSF25. In embodiments, least some autoantibodies, if present in the biological sample, are bound to at least some of the proteins in the plurality of proteins. In embodiments, the autoantibodies that are bound to the proteins, and the composition further comprises detectably labeled antibodies bound to the autoantibodies, such as in any of a variety of immune assays. Thus, in one approach, the disclosure includes determining a signal from the detectably labeled antibodies. In a non-limiting approach, the biological sample is from a cancer patient who has melanoma, but other types of cancers are included, as described further below.

In embodiments, a method of this disclosure comprises comparing the signal to a reference to determine if the biological sample contained autoantibodies from an individual who: i) is likely to exhibit no or mild toxicity from being treated with one or more checkpoint inhibitors; or ii) is likely to exhibit severe toxicity from being treated with one or more checkpoint inhibitors. The method may further comprising determining if the individual is likely exhibit to no, mild or severe toxicity to: iii) treatment with a single checkpoint inhibitor that is an anti-Programmed cell death protein 1 (anti-PD-1) checkpoint inhibitor, and/or iv) treatment with a single checkpoint inhibitor that is an anti-Cytotoxic T-lymphocyte-associated protein 4 (anti-CTLA-4) checkpoint inhibitor, and/or v) treatment with a combination of an anti-PD-1 checkpoint inhibitor and an anti-CTLA-4 checkpoint inhibitor.

In another aspect, the disclosure includes a method comprising treating an individual who has cancer with a checkpoint inhibitor, wherein: a) if the individual is likely to exhibit no or mild toxicity as determined used the above-describe methods, treating the individual with at least one checkpoint inhibitor without an agent that is used to reduce the risk of toxicity from treatment with the checkpoint inhibitor; orb) if the individual is likely to exhibit severe toxicity as determined used the above-describe methods, administering to the individual at least one checkpoint inhibitor, and an agent to reduce the risk of toxicity from the checkpoint inhibitor. In embodiments, the individual is treated with only one checkpoint inhibitor that is an anti-PD-1 checkpoint inhibitor, or the individual is treated with only one checkpoint inhibitor is an anti-CTLA-4 checkpoint inhibitor, or the individual is treated with a combination of the anti-PD-1 and the anti-CTLA-4 checkpoint inhibitors.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All nucleotide sequences described herein include the RNA and DNA equivalents of such sequences, i.e., an RNA sequence includes its cDNA. All nucleotide sequences include their complementary sequences. All protein sequences described herein include all isoforms of such proteins, e.g., proteins made from splice variants, and proteins that may vary from individual to individual in certain amino acids. Thus, all proteins described herein include proteins that have from 90.0-99.9% identity across their entire lengths to such proteins. The amino acid or polynucleotide sequence as the case may be associated with each GenBank accession number of this disclosure is incorporated herein by reference as presented in the database on the effective filing date of this application or patent. This disclosure relates Gowen M F, to J Transl Med. 2018 Apr. 2; 16(1):82. doi: 10.1186/s12967-018-1452-4, from which the Additional Files are incorporated herein by reference.

Aspects of this disclosure include each protein described herein, and all combinations of such proteins, wherein one or more of the proteins are present in vitro and are in contact with a biological sample obtained from an individual who has cancer. In embodiments, the individual from whom a first sample was obtained was not treated with any checkpoint inhibitor before the sample was obtained. In embodiments, the individual from whom a first sample is obtained has been diagnosed with any type of cancer. In embodiments, the cancer is a solid or liquid tumor. In embodiments, the cancer is renal cell carcinoma, breast cancer, prostate cancer, pancreatic cancer, lung cancer, liver cancer, ovarian cancer, cervical cancer, colon cancer, esophageal cancer, stomach cancer, bladder cancer, brain cancer, testicular cancer, head and neck cancer, melanoma or another skin cancer, any sarcoma, including but not limited to fibrosarcoma, angiosarcoma, adenocarcinoma, and rhabdomyosarcoma, and any blood cancer, including all types of leukemia, lymphoma, and myeloma. In a non-limiting embodiment, the biological sample is obtained from an individual who has been diagnosed with melanoma and was not treated with any checkpoint inhibitor before the sample was obtained. In embodiments, a second, third, fourth, sample, etc. can be obtained from an individual who is undergoing treatment and tested to monitor the effect of the treatment, and keep steady, change, adjust, or discontinue treatment with a checkpoint inhibitor. In embodiments, the treatment is adjusted to prevent or mitigate the onset of irAEs, such as by administering an agent to the individual as described further herein.

A method of the present disclosure comprises screening for the presence of one or more sets of antibodies in a biological sample (such as blood, serum, plasma etc.) from an individual who is being considered as a candidate for therapy with immune checkpoint inhibitors, and based upon the antibody profile, identifying the appropriate immune checkpoint inhibitors for administration to the individual, or determining that the individual should not be treated with a checkpoint inhibitor, or determining that the immune checkpoint inhibitors should be administered in conjunction with toxicity mitigation agents/process. The checkpoint inhibitors may be anti-PD-1, anti-CTLA-4, or a combination thereof.

With respect to compositions and methods of this disclosure, antibodies, if present in the biological sample, bind with specificity to one or more proteins that are present in an assay that is designed to determine the presence, absence, and/or amount of such antibodies. Thus, in embodiments, the disclosure comprises exposing a biological sample to a protein array. In embodiments, the protein array comprises at least 50%, 60%, 70%, or 80% of the proteins in the human proteome. In embodiments, the protein array pertains to the proteins known as of the date of the filing of this application or patent. In embodiments, the protein comprise all or a set of proteins present on the Human Proteome Microarray v3.1, commercially available from, for example, CDI NextGen Proteomics. In embodiments, the protein array comprises at least one protein from Table 1. In embodiments, the array comprises two, three, four, five, etc., including all of the proteins described in Table 1, and all combinations thereof. In embodiments, the proteins comprise any one or a combination of proteins under the column “Anti-PD-1”, or the column “Curated Anti-PD-1” or the column “Anti-PD-1+Anti-CTLA-4” or the column “Curated Anti-PD-1+Anti-CTLA-4 (n=25)”, or any combination thereof.

In embodiments, at least one of the proteins on the array used to measure antibodies as described herein is TNFRSF25, the amino acid sequence of which is available from GenBank accession no. NP_683866.1. In this regard, antibodies to TNFRSF25 are significantly different between no/mild and severe toxicity groups. Thus, the present disclosure includes the discovery that a change in anti-TNFRSF25 antibody levels is common to toxicity for all treatment types (CTLA-4, PD-1, and the combination).

In embodiments, the amount of antibodies bound to a proteome array is scored, for example according to the Common Terminology Criteria for Adverse Events (CTCAE). Samples may be divided into groups as further set forth herein. Thus, the disclosure provides compositions and methods for antibody profiling. The antibody profiling may be carried out prior to treatment, any time during the treatment or any time after the treatment. The profiling may be carried out once or multiple times over any period of time.

In one aspect, the present disclosure provides methods for enhancing the efficacy of treatment of cancer, such as melanoma, with immune checkpoint inhibitors. The disclosure also provides panels for detection of subsets of antibodies that can form a basis for treatment decisions in the treatment of cancer, such as melanoma. The disclosure also provides kits for detection of specific antibodies.

In embodiments, a method of this disclosure comprises: a) obtaining a sample of a biological sample, such as blood, plasma or serum, b) determining antibodies using a protein array; and c) based on the profile of the antibodies, determining that the individual is not a candidate for a checkpoint inhibitor, or administering one or more immune checkpoint inhibitors to the individual. The method can further comprise administering to the individual agents to mitigate expected or observed toxicity from the checkpoint inhibitors. The set of antibodies to be screened can be one or more, or all of the antibodies, that bind to the proteins described herein. In embodiments, the antibodies are specific for proteins listed Table 1. In embodiments, the antibodies are specific for proteins listed in Table 1 under the column “Anti-PD-1.” In embodiments, the antibodies are specific for proteins listed in Table 1 under the column “Curated Anti-PD-1.” In embodiments, the antibodies are specific for proteins listed in Table 1 under the column “Anti-PD-1+Anti-CTLA-4.”

TABLE 1

| Anti-PD-1 | NCBI GenBank RefSeq | Curated Anti- |
|---|---|---|
| (n = 40) | Protein Accession Number | PD-1 (n = 28) |
| AP2M1 | NP_001298127.1 | AP2M1 |
| C11orf71 | NP_061894.2 | C18orf8 |
| C18orf8 | NP_037458.3 | C7orf25 |
| C7orf25 | NP_001093328.1 | CBR3 |
| CBR3 | NP_001227.1 | CSNK1D |
| CD247 | NP_932170.1 | CTTNBP2NL |
| CSNK1D | NP_001350678.1 | EDDM3A |
| CTTNBP2NL | NP_061174.1 | EIF2AK3 |
| EDDM3A | NP_006674.2 | EIF4EBP3 |
| EIF2AK3 | NP_004827.4 | ELAVL3 |
| EIF4EBP3 | NP_003723.1 | FAM107B |
| ELAVL3 | NP_001411.2 | GDNF |
| EML1 | NP_001008707.1 | LSM8 |
| FAM107B | NP_001269624.1 | MED6 |
| FOXP4 | NP_001012426.1 | MROH8 |
| GDNF | NP_001177397.1 | PLS3 |
| HIST1H2BC | NP_003517.2 | POLK_frag |
| LSM8 | NP_057284.1 | RBMS3 |
| MED6 | NP_001271138.1 | RPS21 |
| MROH8 | NP_689716.4 | SCO2 |
| NDUFS1 | NP_004997.4 | SESN1 |
| ORC4L | NP_001177808.1 | SH3BGRL2 |
| PLS3 | NP_005023.2 | SLC10A5 |
| POLK_frag | NP_057302.1 | SLC29A4 |
| PSMA6 | NP_002782.1 | SLTM |
| RBMS3 | NP_001003793.1 | SPC25 |
| RPS21 | NP_001015.1 | TNFRSF25 |
| SCO2 | NP_001162580.1 | ZMYM5 |
| SESN1 | NP_055269.1 | |
| SH3BGRL2 | NP_113657.1 | |
| SKIL | NP_005405.2 | |
| SLC10A5 | NP_001010893.1 | |
| SLC29A4 | NP_001035751.1 | |
| SLTM | NP_079031.2 | |
| SOAT2 | NP_003569.1 | |
| SPC25 | NP_065726.1 | |
| TNFRSF25 | NP_683866.1 | |
| USP25 | NP_001269970.1 | |
| YIF1B | NP_001034761.1 | |
| ZMYM5 | NP_001034739.1 | |

| Anti-PD-1 + | |
|---|---|
| Anti-CTLA-4 | |
| (n = 37) | NCBI GenBank RefSeq Protein Accession Number |
| AGL | NP_000019.2 |
| AP3D1 | NP_001248755.1 |
| AZU1 | NP_001691.1 |
| CDC23 | NP_004652.2 |
| DHDH | NP_055290.1 |
| FAM3A | NP_001164603.1 |
| FGFR1OP2 | NP_056448.1 |
| FKBP2 | NP_004461.2 |
| FUZ | NP_079405.2 |
| GRB2 | NP_002077.1 |
| GRM3 | NP_000831.2 |
| HAUS2 | NP_060567.1 |
| HG497681.1_frag | CDI clone: JHU10789 |
| | Protein sequence: |
| | MLQPLQESGIIMEQALRKNRLQLGTEQPGCTPDASGTWCLLW |
| | RMGQLPHCPGARASDPGAKVCLFHFWELAVFARLSGPQASHC |
| | PPGITFLQDHGEDDMRC (SEQ ID NO: 1) |
| HR | NP_005135.2 |
| ING3 | NP_061944.2 |
| IPO11 | NP_001128251.1 |
| KJ902887_frag | CDI clone: JHU08536 |
| | MQCLLPYQSKEPSCLPPLPLNLPLPPCLCPLLQLNAAMTRKE |
| | KTKEGQRAAQFSAGADAGSGGGLSRQKDTKRPMLLVIHDVVL |
| | ELLTSSDCHANPRKYPTCQKSEVLGVSIYVSICPSTRPRDKN |
| | KTKKRCQVLEAVLVSKPSGSCHQGSFEIVPHVKGNLAFTSSN |
| | H (SEQ ID NO: 2)_ |
| KLRG1 | NP_001316028.1 |
| LAMP2 | NP_002285.1 |
| LY6G6C | NP_079537.1 |
| MARK4 | NP_001186796.1 |
| N6AMT1 | NP_037372.4 |
| OGG1 | NP_002533.1 |
| PIKFYVE | NP_055855.2 |
| PRKD2 | NP_057541.2 |
| RBM7 | NP_001272974.1 |
| RFC5 | NP_031396.1 |
| RP11- | CDI clone: JHU03224 |
| 998D10.4_frag | MFHQILVGLKKHSSFIPLRIYEIRRYWSSAVCPASGIVQSRC |
| | (SEQ ID NO: 3) |
| SEC23B | NP_116780.1 |
| SIRT6 | NP_001180214.1 |
| SNX9 | NP_057308.1 |
| TCTN2 | NP_079085.2 |
| TMEM178A | NP_689603.2 |
| TNFRSF25 | NP_683866.1 |
| USP36 | NP_001308220.1 |
| VASP | NP_003361.1 |
| ZBTB44 | NP_001288027.1 |

Curated Anti-PD-1 + Anti-CTLA-4 (n = 25)
AGL
AP3D1
DHDH
EPB41L3
FAM3A
FKBP2
FUZ
GRB2
HAUS2
HG497681.1_frag
HR
IPO11
KJ902887_frag
KLRG1
LY6G6C
MARK4
PIKFYVE
RBM7
SEC23B
SIRT6
SNX9
TCTN2
TMEM178A
VASP
ZBTB44
In Table 1, “CDI clone” refers to amino acid sequences available from: https://collection.cdi-lab.com/public/clones/3226

Change(s) in the levels of these antibodies compared to a reference level, are indicative the particular level or type of toxicity listed for that column.

Data analysis provided herein indicates that the presence and/or amount of antibodies from Table 1 under one or both of the columns “Anti-PD-1” or “Curated Anti-PD-1” permits distinguishing between the likelihood of severe toxicity versus no or mild toxicity for treatment with anti-PD-1. Thus in one embodiment, the presence or absence or amount of one or more antibodies that recognize proteins from these columns is determined in a biological sample from an individual (e.g., blood, serum or plasma) prior to start of treatment. Thus, the disclosure provides for distinguishing an individual as someone likely to exhibit severe toxicity to anti-PD-1 antibody therapy compared to an individual who is likely to exhibit no or mild toxicity. Likewise, the disclosure includes determining the presence and/or amount of antibodies that recognize proteins from Table 1 under the column “Anti-PD-1+Anti-CTLA-4” to thereby distinguishing an individual as someone likely to exhibit severe toxicity to the combination therapy, relative to an individual who is likely to exhibit no or mild toxicity to the combination therapy. In embodiments, the disclosure also includes distinguishing an individual as someone likely to exhibit mild or severe toxicity for CTLA-4 antibody therapy compared to an individual who is likely to exhibit no toxicity.

In one embodiment, the presence or absence of all the antibodies in Table 1 is determined prior to start of treatment with an immune checkpoint inhibitor. In embodiments, the presence or absence of all the antibodies in Table 1 is tested during the course of treatment with an immune checkpoint inhibitor to monitor the development of irAEs, and adjust treatment if needed.

The amount of antibodies, or a change in the level of antibodies, means a level that is measured against a suitable reference, such as a reference value. The reference may be established from a population of relevant individuals from which group the distinction is to be made. For example, the reference can be an average value from a group of individuals who have not shown toxicity, shown mild toxicity, or shown severe toxicity to the particular treatment. These values could be used as references for no toxicity, mild toxicity or severe toxicity, or site-specific toxicity. Other references can be obtained in a similar manner. For no toxicity, individuals who have not been treated at all may also be used.

The presence of antibodies in a patient sample can be detected by methods that are known in the art. For example, any type of immunological assay or antigen binding assay may be used. A commonly used assay is ELISA. Detection of the antigen-antibody complex is generally done by using detectable (fluorescent, luminescent, chemiluminescent, radioactive etc.) labels.

In one aspect, this disclosure provides kits for aiding in enhancing the efficacy of treatment with CTLA-4 antibodies and PD-1 antibodies. The kit may comprise a complete set of detection agents for antibodies that bind with specificity to the proteins listed in each column of Table 1. The reagents may comprise proteins, or antigenic fragments thereof, which may be immobilized on a substrate, detectable molecules for detecting antigen-antibody binding, buffers etc.

The present biomarker assay can be used to guide the clinical management of melanoma patients. For example, it could be used alone or in combination with a predictive test of immunotherapy efficacy to inform clinicians as to the most appropriate type of therapy (e.g. anti-PD-1 vs anti-CTLA-4 vs combination).

There appears to be minimal overlap in toxicity-associated antibodies between different immunotherapies (i.e. anti-PD-1 vs. anti-CTLA-4), and therefore, a toxicity biomarker test might reveal that a patient is susceptible to developing severe toxicity from one therapy but not from another—in which case, the appropriate and individualized therapy can be selected for that individual.

In one embodiment, a patient who is predicted to develop severe toxicity (or severe toxicity affecting specific organ/tissue sites or likely requiring treatment termination) could be monitored for the development of toxicity, or could be treated with a different dosage of immune checkpoint inhibitor(s). Such monitoring could allow clinicians to intervene e.g. with steroids, to mitigate toxicity (immune related adverse events) as they develop.

In one embodiment, the present methods can also be used in adjuvant immune checkpoint blockade in earlier stage (3 or even 2) melanoma as being able to identify patients at risk of severe toxicity would be especially beneficial in the adjuvant setting, where there is less tolerance for severe toxicity.

If an indication of likelihood of toxicity is observed, then steps can be taken to mitigate the toxicity, or the treatment regimen of anti-CTLA-4 and/or anti-PD-1 can be interrupted or the dose reduced.

For mitigating toxicity, corticosteroid treatment may be administered. For example, prednisone may be administered orally or via i.v. For skin rashes, topical corticosteroids may be used.

Another approach is to administer a tumor necrosis factor-alpha (TNF-α) inhibitor prior to or concurrent with one or a combination of immune checkpoint inhibitors. A non-limiting embodiment of a suitable TNF-α inhibitor is infliximab (a product sold under the brand name REMICADE®), but other TNF-α inhibitors may also be used. Non-limiting examples of other suitable TNF-α inhibitors include Infliximab-abda (a product sold under the brand name RENFLEXIS®), Infliximab-dyyb (a product sold under the brand name INFLECTRA®), Adalimumab (a product sold under the brand name HUMIRA®), Adalimumab-adaz (a product sold under the brand name), Adalimumab-atto (a product sold under the brand name AMJEVITA®), Certolizumab pegol (a product sold under the brand name CIMZIA®), Etanercept (a product sold under the brand name ENBREL®), Etanercept-SZZS (a product sold under the brand name EREIZI®), and Golimumab (a product sold under the brand names SIMPONI® and SIMPONI ARIA®). Other treatments for steroid-refractory irAEs—typically colitis—include: mycophenolic acid, or tacrolimus.

For individuals with opportunistic infections, trimethoprim-sulfamethoxazole, atovaquone, or pentamindine may be used. For pruritus, oral antipruritics (e. g, hydroxyzine, diphenhydramine may be used. For diarrhea or colitis, infliximab or mycophenolate may be used. For hepatic toxicity, mycophenolate may be used. For pneumonitis, infliximab with or without cyclophosphamide maybe used. For other toxicities, generally a combination of one or more of the above may be used. Infliximab-refractory cases may be treated with mycophenolate or vedolizumab and tacrolimus.

In some embodiments, the following methods are provided: A method comprising contacting a human proteome array with a biological sample and determining antibody binding to the array, and based on determining the binding identifying the individual as having no/mild or severe toxicity, wherein the identifying is optionally performed using a score for Common Terminology Criteria for Adverse Events (CTCAE), v5.0CTCAE scoring criteria and methods are known in the art and can be readily adapted for use in embodiments of the present disclosure. As is known in the art, for CTCAE grading, no/mild toxicity is assigned a grade of 0-2, severe is assigned a grade 3 or higher.

In embodiments, a CTCAE score may comprise a range of grades of toxicity for any condition, disease or disorder for which a CTCAE score would be determined, and such will be understood by those skilled in the art. In this regard, checkpoint inhibitor treatment is known in the art to be associated with irAEs that can be transient but also can be severe or fatal. The most common and clinically important irAEs are dermatologic, diarrhea/colitis, hepatotoxicity, and endocrinopathies, although other sites can also be affected. Thus, in embodiments, a CTCAE score is calculated related to a condition, disease or disorder of the skin, or the gastrointestinal (GI) system, or the kidney, or the pancreas, or the nervous system, or the eye, or the blood, or the organs or tissues of the cardiovascular system, or the immune system, or rheumatologic and musculoskeletal or are endocrinopathies, or combinations thereof.

In view of the above-described similarities in clinical presentation between patients experiencing irAEs from ICI therapy and those with autoimmune disorders, such as colitis, hepatitis, thyroiditis, nephritis, hypophysitis, rashes and arthralgias [16], the present disclosure provides an analysis that was designed to determine if a subset of melanoma patients have a baseline (pre-treatment) autoimmune susceptibility, characterized by a repertoire of preexisting autoantibodies against specific antigen targets, which can predict development of irAEs following ICI therapy. This was tested using a human proteome microarray to identify toxicity-associated autoantibodies in pre-treatment sera from 75 metastatic melanoma patients who received anti-CTLA-4, anti-PD-1, or combination treatment (anti-CTLA-4 and anti-PD-1 together).

The following examples are provided as illustrative of the present methods. These examples are not intended to be restrictive in any way.

### Examples

Reproducibility of a proteomic microarray for serum antibody profiling.

We assessed the intra-chip and inter-chip reproducibility of a human proteome microarray (HuProt v3.1, CDI Labs) using pre-treatment sera from a cohort of 10 metastatic melanoma patients Table 2.

TABLE 2
Table 2. Summary of clinical features from independent group of
10 melanoma patients treated with anti-CTLA-4 (n = 3), anti-
PD-1 (n = 3), or combined anti-CTLA-4/anti-PD-1 (n =
4), and from whom serum samples were used to assess assay reproducibility.

| | | anti-CTLA-4 | | anti-PD-1 | | combination | |
|---|---|---|---|---|---|---|---|
| | | (n = 3) | | (n = 3) | | (n = 4) | |
| | | No. (%) | | No. (%) | | No. (%) | |
| Gender | Female | 2 | (67) | 1 | (33) | 3 | (75) |
| | Male | 1 | (33) | 2 | (67) | 1 | (25) |
| Age at | Mean (SD) | 55.5 | (5.58) | 72.65 | (17.4) | 67.1 | (4.37) |
| Treatment Initiation | Median | 56 | | 82.2 | | 66.9 | |
| ECOG PS (pre- | 0 | 3 | (100) | 3 | (100) | 3 | (75) |
| treatment) | >1 | 0 | | 0 | | 1 | (25) |
| LDH (pre- | Normal | 3 | | 0 | | 4 | (100) |
| treatment) | Elevated | 0 | | 3 | (100) | 0 | |
| | Unknown | 0 | | 0 | | 0 | |
| Response to | POD | 0 | | 0 | | 0 | |
| treatment | SD | 2 | (67) | 2 | (67) | 0 | |
| | PR | 1 | (33) | 1 | (33) | 3 | (75) |
| | CR | 0 | | 0 | | 1 | (25) |
| | UNC | 0 | | 0 | | 0 | |
| Toxicity | None | 0 | | 0 | | 0 | |
| | Mild | 2 | (67) | 1 | (33) | 1 | (25) |
| | Severe | 1 | (33) | 2 | (67) | 3 | (75) |
| GI Toxicity | Mild | 1 | (33) | 2 | (67) | 1 | (25) |
| | Severe | 2 | (67) | 1 | (33) | 2 | (50) |
| Skin Toxicity | Mild | 3 | (100) | 1 | (33) | 3 | (75) |
| | Severe | 0 | | 0.0 | | 0.0 | |
| Endocrine | Mild | 1 | (33) | 1 | (33) | 1 | (25) |
| Toxicity | Severe | 0 | | 0.0 | | 1 | (25) |
| Required | Yes | 0 | | 1 | (33) | 0 | |
| Treatment | No | 3 | (100) | 2 | (67) | 4 | (100) |
| Termination | | | | | | | |

LDH, lactate dehydrogenase; POD, progression of disease; SD, stable disease; PR, partial response; CR, complete response; UNC, unclassified.

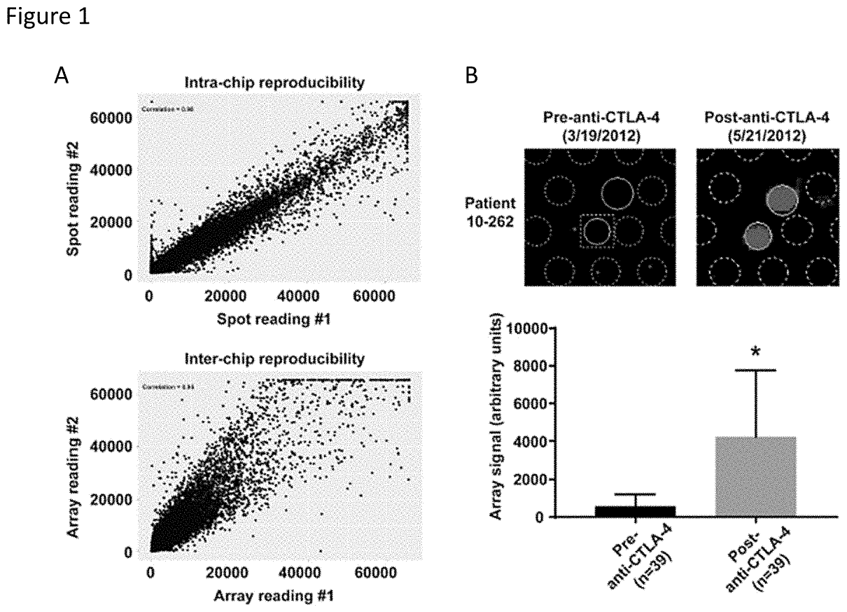

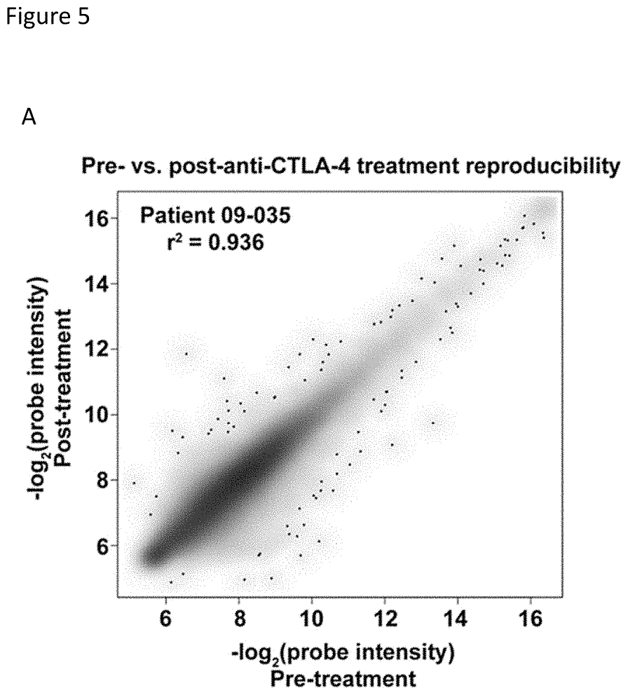

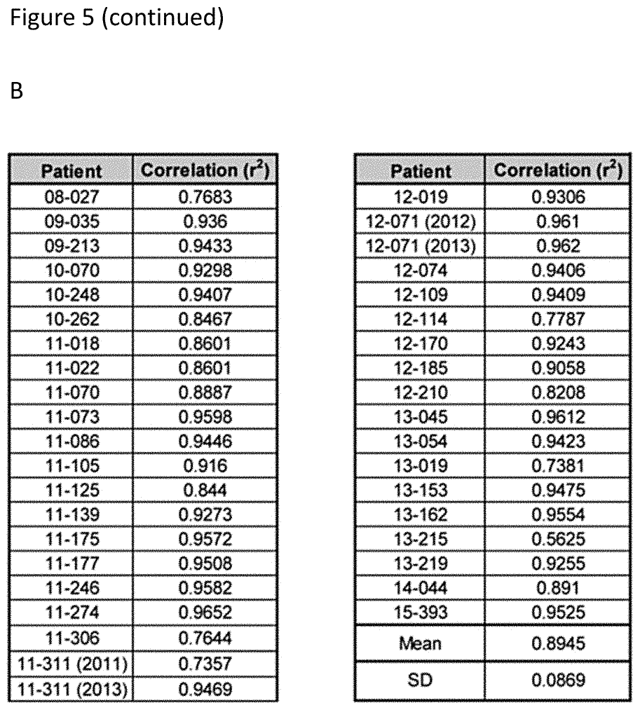

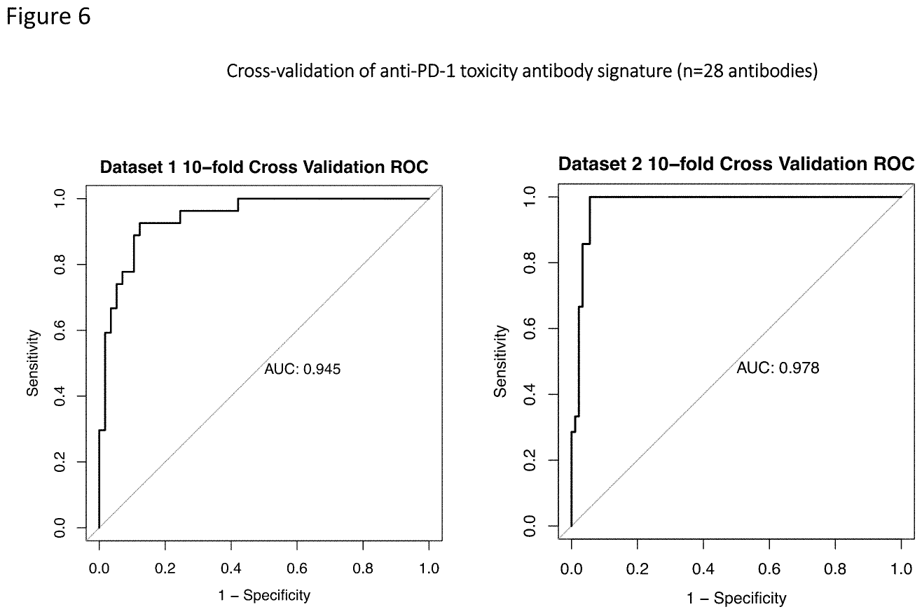

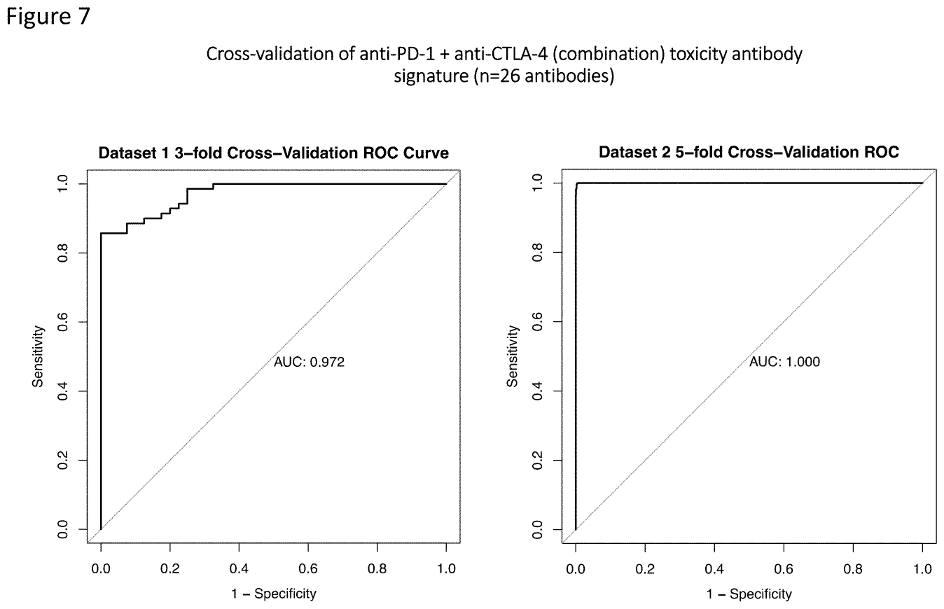

We assessed the correlation between duplicate immunoglobulin G (IgG) spots on each chip and found a high degree of intra-chip reproducibility (r²=0.98; FIG. 1A, top). The same 10 sera samples were also assayed on two separate occasions to assess inter-chip reproducibility, which showed a strong correlation between IgG antibody readings across chips (r²=0.95; FIG. 1A, bottom). We then tested anti-CTLA-4 IgG antibody levels between matched pre- and post-treatment sera from an independent anti-CTLA-4 cohort (n=39 samples) as an internal control, and found that anti-CTLA-4 IgG antibody levels were significantly increased in post-treatment vs. pre-treatment sera (p<0.0001; FIG. 1B). Our analysis also showed a strong correlation (mean r²=0.89) between global IgG antibody levels from pre- and post-anti-CTLA-4 treatment sera (FIG. 5). Hence, the human proteome microarray allows reproducible and sensitive profiling of serum autoantibodies, making it suited to identification of differences in pre-treatment autoantibody levels in patient sera.

Differences in baseline serum autoantibodies predict development of immunotherapy toxicity.

To analyze whether a specific baseline autoantibody profile can predict development of toxicity following treatment with ICI, we assessed IgG antibody levels in 78 baseline serum samples from 75 ICI-treated metastatic melanoma patients. We assayed 39 serum samples from 37 anti-CTLA-4-treated patients, 28 serum samples from 27 patients treated with anti-PD-1, and 11 samples from 11 patients treated with combined anti-CTLA-4/anti-PD-1

The severity of immune toxicity was graded according to objective Common Terminology Criteria for Adverse Events (CTCAE), following detailed review of patient medical records by a single investigator (MW), as either no toxicity (grade 0), mild toxicity (grade 1-2) or severe toxicity (grade 3-4). We also noted the location and type of immune toxicity (gastrointestinal, skin, endocrine) experienced by each patient. Comparing patients treated with anti-CTLA-4, anti-PD-1, or combined anti-CTLA-4/anti-PD-1, there was no statistically significant difference in gender, age at treatment initiation, pre-treatment lactate dehydrogenase (LDH) levels, or pre-treatment Eastern Cooperative Oncology Group Performance Status (ECOG PS; [19]) (Table 1). Furthermore, we did not observe significant differences in the severity or location of toxicity between treatment groups. Compared to anti-CTLA-4 or anti-PD-1 monotherapy patients, the combination treatment cohort showed significantly better response to therapy (p=0.01) but also significantly more treatment termination (p=0.006), which is consistent with clinical trials demonstrating the greater efficacy and increased toxicity with combined ICI [2].

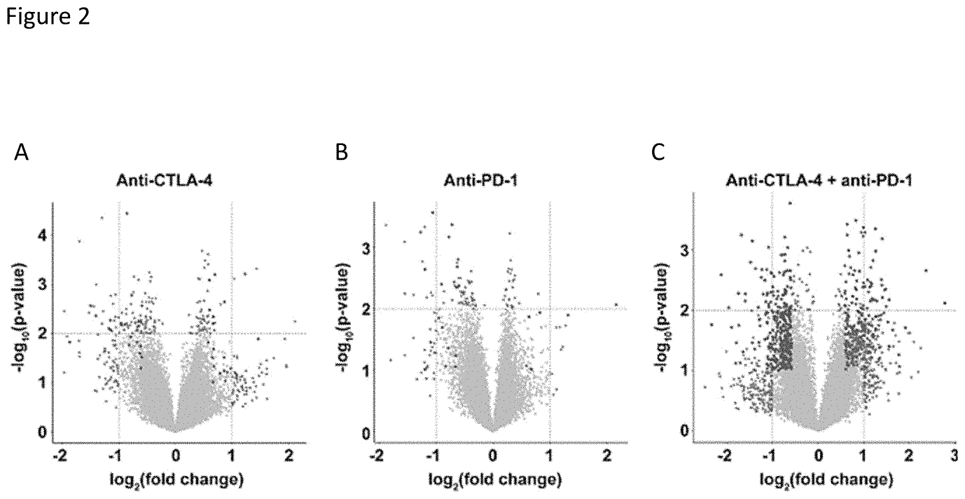

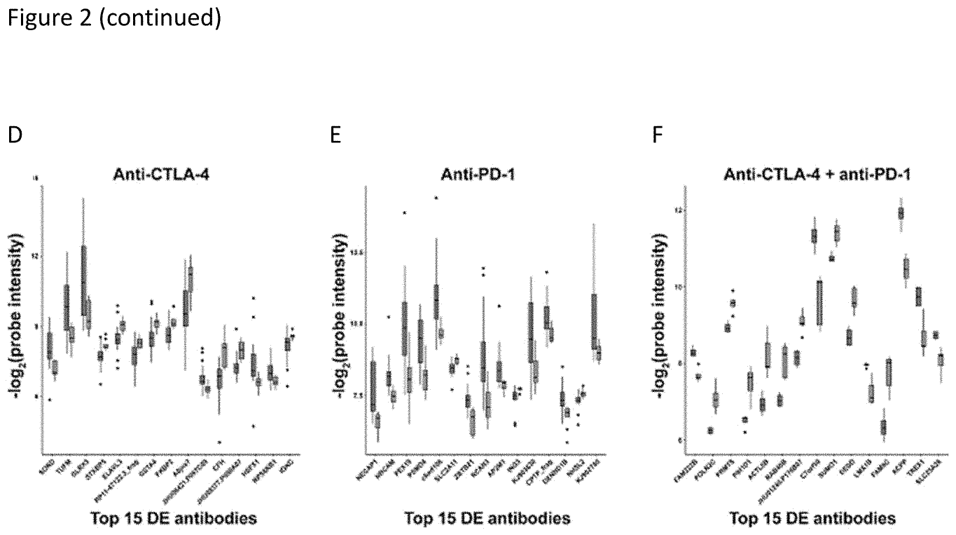

To identify pre-immunotherapy toxicity-associated autoantibodies, we compared IgG autoantibody profiles between anti-CTLA-4- or anti-PD-1-treated patients who experienced no or mild vs. severe toxicity. For pre-treatment samples from the combined anti-CTLA-4 and anti-PD-1 treatment group, we compared IgG antibodies between mild and severe toxicity samples, as all patients developed some degree of immune-related toxicity with this regimen. We observed toxicity-associated differences in IgG antibody levels for each ICI treatment (FIG. 2A, 2B, 2C), and set two thresholds for differential antibody expression for each comparison based on power calculations derived from experimental data. Differentially expressed (DE) antibodies were defined as those with p-value <0.05 between no/mild and severe toxicity (FIG. 2D, 2E, 2F). We identified 914 DE antibodies associated with severe toxicity in the anti-CTLA-4 cohort, 723 DE antibodies associated with severe toxicity in the anti-PD-1 cohort, and 1,161 DE antibodies associated with severe toxicity in the combination treatment cohort

TABLE 3
Table 3. Numbers of differentially expressed (DE), strongly differentially
expressed (strong DE), filtered and curated antibodies are shown for
comparisons of none/mild vs. severe toxicity, across three different
treatment groups (anti-CTLA-4, anti-PD-1, and combination).

| | No. | No. | No. |
|---|---|---|---|
| Comparison | DE¹ | Filtered² | Curated³ |
| Anti-CTLA-4 -None/Mild vs. Severe | 914 | 519 | 45 |
| Anti-PD-1 -None/Mild vs. Severe | 723 | 221 | 25 |
| Anti-CTLA-4 + anti-PD-1 - | 1161 | 1344 | 575 |
| Mild vs. Severe | | | |

¹p-val < 0.05
²p-val < 0.01 and |log₂ (FC)| > log₂ (1.5)
³Selected by information gain

We observed a minimal degree of overlap in toxicity-associated IgG antibodies (DE) between monotherapy groups (antiCTLA-4 or anti-PD-1) and the combination therapy (anti-CTLA-4+anti-PD-1) group. For example, there were only 99 IgG antibodies in common between 849 unique anti-CTLA4 toxicity-associated IgG antibodies and 1,071 unique anti-CTLA-4 and anti-PD-1 toxicity-associated antibodies. Similarly, there were only 54 IgG antibodies in common between 683 unique anti-PD-1 toxicity-associated IgG antibodies and 1,071 unique anti CTLA-4 and anti-PD-1 toxicity-associated antibodies (data not shown). This suggests that discrete, treatment type-specific sets of antibodies are associated with ICI toxicity.

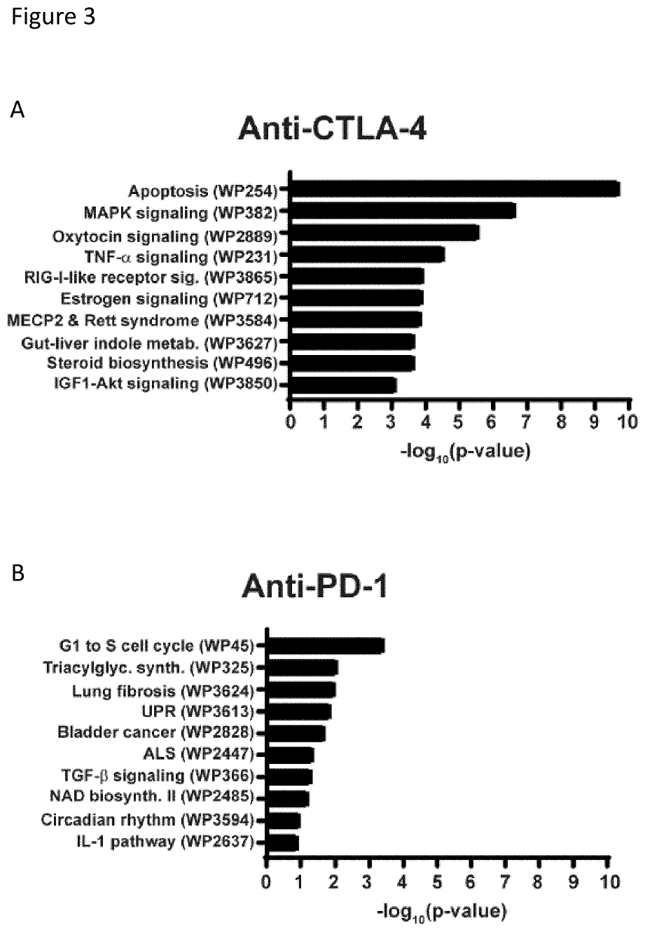

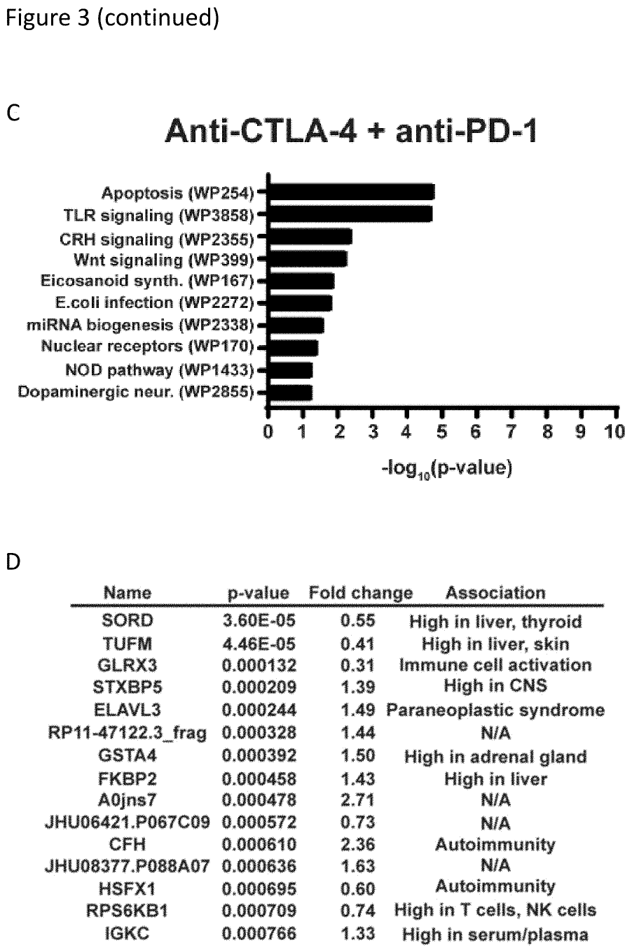

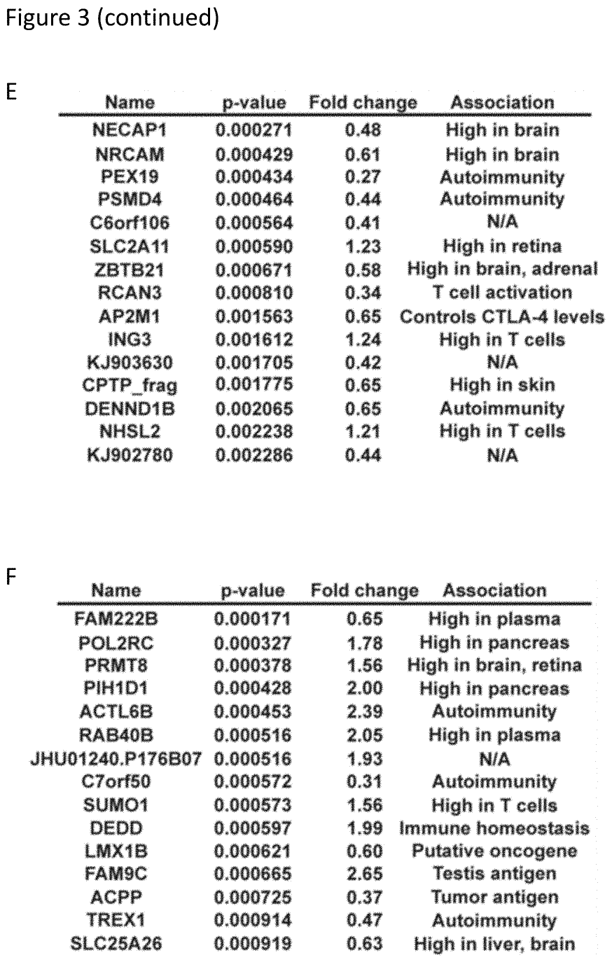

To analyze potential causative roles for toxicity-associated antibodies in development of irAEs, we performed pathway analysis on the protein antigen targets identified for each treatment group. We elected to focus our analysis on the filtered sets of toxicity-associated antibodies for each treatment type, as defined above. Our results revealed significant enrichment of proteins in pathways that have been previously associated with immunity/autoimmunity, including “Apoptosis”, “TNF-α signaling”, “Lung fibrosis”, “IL-1 pathway”, “Toll-like receptor (TLR) signaling”, “E. coli infection”, and “microRNA biogenesis” (FIG. 3A, 3B, 3C). A literature analysis for the fifteen most DE toxicity-associated antibodies for each treatment group (FIG. 2D, 2E, 2F) revealed that their protein targets were highly expressed in tissues that are commonly affected in patients experiencing irAEs, including liver and skin, and have been implicated in the regulation of immune cell activity and in autoimmune disorders (FIG. 3D, 3E, 3F). Together, the data indicate that a subset of toxicity-associated antibodies could not only highlight patients at risk of irAEs from immunotherapy, but might also play a causative role in the development of immune toxicity.

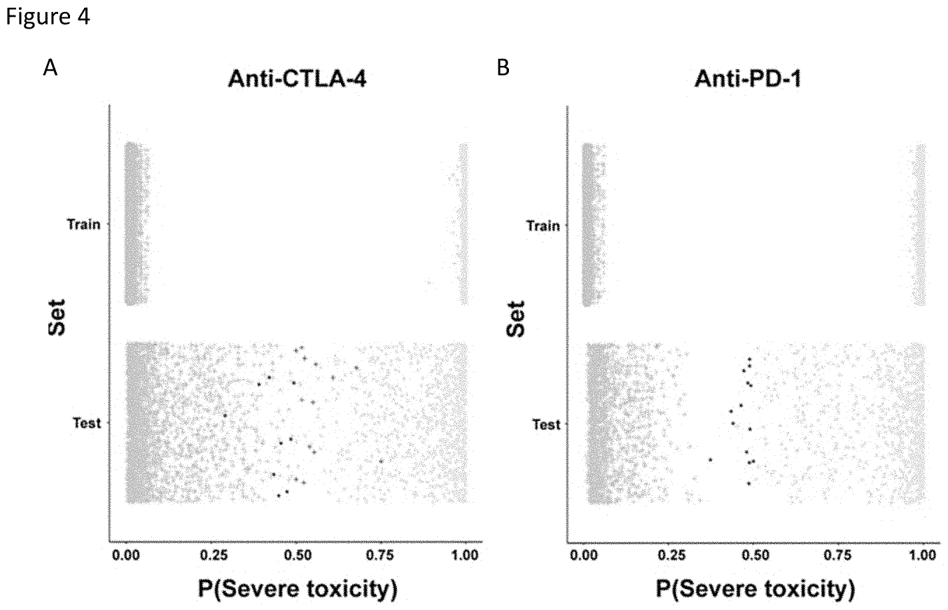

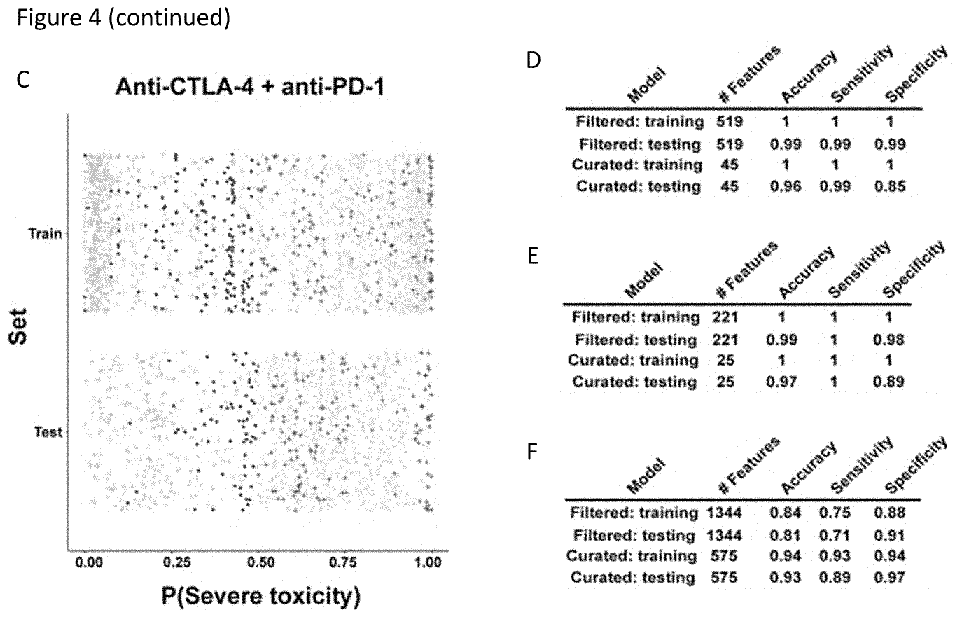

To develop an approach to predict toxicity development in melanoma patients treated with ICI, we derived support vector machine (SVM) classification models to classify patients according to their risk of developing severe immunotherapy-related toxicity based on the levels of specific antibodies (features) in baseline sera. We performed SVM model training and testing for each treatment group using “filtered’ and “curated” feature lists (as defined above). For each model, we used 3- (combination therapy) or 5-fold (monotherapy) cross-validation and repeated the scheme 100 times to mitigate the impact of overfitting (FIG. 4A, 4B, 4C). “Filtered” antibody sets predicted severe toxicity development with excellent (>0.98) accuracy, sensitivity, and specificity for antiCTLA-4 (FIG. 4D) and anti-PD-1 (FIG. 4E) monotherapy groups, and with good (>0.71) accuracy, sensitivity, and specificity for the smaller group of combined anti CTLA-4 and anti-PD-1 patients (FIG. 4F). The prediction models we derived using the smaller “curated” antibody sets (n=45 for anti-CTLA-4, n=25 for anti-PD-1, n=575 for combination treatment) showed very good (>0.85) accuracy, sensitivity, and specificity for all three treatment groups (FIG. 4D, 4E, 4F). These results suggest that baseline antibody signatures should be evaluated further for their clinical utility as biomarkers to predict toxicity from immunotherapy.

The following materials and methods were used to produce the results of this disclosure.

Study Population and Serum Collection

Metastatic melanoma patients treated with ICI therapy at New York University (NYU) Langone Health from 2011 to 2016 were enrolled in the Interdisciplinary Melanoma Cooperative Group (IMCG) biospecimen database protocol. This protocol, approved by the NYU Institutional Review Board, prospectively enrolls patients with melanoma presenting to surgical and medical oncologists at the NYU Perlmutter Cancer Center (PCC), and banks patient biospecimens (linked to extensive, prospectively recorded clinicopathological data) for research purposes with protocol-driven follow up every 3 months [17]. Informed consent for use of clinical data and specimens was obtained from all patients at the time of enrollment.

To minimize pre-analytical variability, samples were routinely collected, processed, and stored using standardized NYU IMCG protocols. Blood was collected in Becton Dickinson Vacutainer SST Venous Blood Collection—Serum tubes (catalog #366430). For consistency and reproducibility, samples were processed <90 minutes after collection by centrifugation for 10 minutes at 2,500 rpm at room temperature. Aliquots (1 ml) of sera were stored in 1.8 ml cryovials at −80 C, and thawed once at the time of the proteomic array assay.

For assay validation purposes, two identical serum samples were collected from 10 immunotherapy treated patients: (i) anti-CTLA-4 (n=3), (iii) anti-PD-1 (n=3), and (iii) combination therapy (n=4), to assess the reproducibility of the proteomic microarray. All sera were aliquoted into smaller volumes and stored at −80° C. until further use, and thawed on ice prior to the assay.

Pre-treatment sera samples (n=78) were prospectively collected from three different ICI-based cohorts of stage IV metastatic melanoma patients: (i) anti-CTLA-4 (n=39 samples from 37 patients), (ii) anti-PD-1 (n=28 samples from 27 patients), and (iii) anti-CTLA4/anti-PD-1 combination therapy (n=11 samples from 11 patients). Patient-matched post-treatment samples were also collected for the anti-CTLA-4 cohort. Samples were grouped based on immunotherapy toxicity outcomes that were determined from treatment initiation to at least 6 months after the last treatment. Clinicians treating patients at the NYU PCC rigorously assessed toxicity according to objective Common Terminology Criteria for Adverse Events (CTCAE) criteria. All patient medical records underwent additional review by a medical oncologist (MW) to account for differences in toxicity monitoring of patients treated on and off protocol. Toxicity was stratified into three clinically-relevant groups: no toxicity (CTCAE grade 0), mild toxicity (CTCAE grade 1-2), and severe toxicity (CTCAE grade 3-4).

Serum Antibody Profiling Using a Human Proteome Microarray

To profile serum antibodies, we utilized a human proteome microarray (HuProt Human Proteome Microarray v3.1, CDI Labs, Mayaguez, PR) that contains over 19,000 unique, individually-purified full-length human proteins in duplicate, covering more than 75% of the proteome [18]. Briefly, the HuProt arrays were blocked with blocking solution (5% BSA/1×TBS-T) at room temperature for 1 hour, and then probed with serum samples (diluted 1:1,000) at 4° C. overnight. The arrays were then washed with 1×TBS-T for 3 times, 10 mins each, and probed with Alexa-647 labeled anti-human IgG (Jackson ImmunoResearch, West Grove, Pa.) at room temperature for 1 hour, followed by 3 washes of 1×TBS-T, 10 mins each, and then spun to dryness prior to scanning.

Array Data Pre-Processing

Slides were scanned using a GenePix 4000B instrument (Molecular Dynamics, Sunnyvale, Calif.) and GenePix Pro (v7.2.22) software was used to measure the signal intensities for IgG binding to array features as well as any background signal present. Before pre-processing, each array was manually inspected and problematic probes were flagged. For each sample array, resulting GPR files were processed using the Bioconductor (v3.5) package PAA (v1.10.0) in R (v3.4.1).

To assess the overall quality of individual arrays, foreground and background signal intensities were plotted by array position to determine any regions containing technical artifacts. These regions were noted and compared to array plots made following all preprocessing to assess the cumulative effect of all procedures on individual arrays. The signal intensities for probes which had been manually flagged were replaced by the median signal intensity for all probes which were not flagged, and arrays were subsequently corrected for background intensities using the Bioconductor package limma (v3.32.5) with the “normexp” model and a saddle-point approximation. To determine the appropriate normalization procedure, MA plots were created for each sample array and the effects of cyclic loess, quantile, and vsn normalization visualized. Cyclic loess normalization gave the best normalization across all arrays and was applied using the normalizeArrays function in the PAA package. Finally, a combined signal intensity was generated from the duplicate probes for each antibody using the mean of the individual signal intensities and changing to log₂ scale.

Analysis of Differential Levels of Serum Antibodies

For each treatment type, pre-treatment samples were assigned to one of two toxicity groups (no/mild toxicity versus severe toxicity) for differential expression analysis. Student's t-Test was used to determine if there was a significant difference between average signal intensities for each antibody across toxicity groups, and p-values and log₂ fold change (FC) were recorded for each antibody. The power calculations for comparing the toxicity groups for the three treatments are shown in Table 4, and indicate that the studied sample sizes are adequately powered (>=80%) to detect antibodies with FCs at 1.15, 1.13 and 1.48 at alpha=0.01 for the anti-CTLA-4, anti-PD-1, or combination treatments, respectively. Antibodies with p value <0.05 between toxicity groups were defined as being differentially expressed (DE), and those with p-value <0.01 and |log₂(FC)|>log₂(1.5) were designated as belonging to a “filtered” list of DE antibodies associated with toxicity. Both DE and “filtered” antibodies were used in further analyses.

TABLE 4

| Treatment | | | | SD of the | | Detectable |
|---|---|---|---|---|---|---|
| Group | Alpha | N1 | N2 | difference in log-exp | COV* | FC* |
| anti-CTLA- | 0.01 | 9 | 30 | 0.1291 | 13% | 1.152 |
| 4 | 0.05 | 9 | 30 | 0.1291 | 13% | 1.193 |
| anti-PD-1 | 0.01 | 9 | 19 | 0.1024 | 10% | 1.125 |
| | 0.05 | 9 | 19 | 0.1024 | 10% | 1.159 |
| combination | 0.01 | 4 | 7 | 0.1507 | 15% | 1.483 |
| treatment | 0.05 | 4 | 7 | 0.1507 | 15% | 1.342 |

*calcuated using the relationship between the means and variances of Y and X = log(Y): COV(Y) = √[Exp{σ(X)²} − 1].
*PASS 14 Power Analysis and Sample Size Software (2015). NCSS, LLC. Kaysville, Utah, USA, ncss.com/software/pass.

Classification Models for Treatment Toxicity

For each treatment type and each antibody in the “filtered” differentially expressed list, the Shannon entropy was calculated and information gain derived. Information gain describes how important a particular feature (antibody) is with regards to the model being developed. Any antibodies with corresponding information gain >0.05 were retained as a part of a “curated” antibody feature set. While this threshold is low, it was necessary due to the relatively low number of samples available and still enables the identification of antibodies involved in toxicity prediction.

Using the “filtered” and “curated” antibody sets separately, two support vector machine (SVM) classification models were built using R package e1071 (v1.6.8) with type parameter C-classification and radial bias kernel. For each model, samples were divided into either 3 or 5 folds, depending on the number of samples available in each toxicity group, and cross-validation used to assess model performance. Each fold was left out for testing once, and a model trained using the remaining folds. Every model was evaluated for training and testing accuracy, sensitivity, and specificity, and for each sample the probability of being designated severe toxicity was recorded. Samples with no/mild toxicity were designated as “negative” and those with severe toxicity designated as “positive”; therefore, sensitivity describes the proportion of severe toxicity samples accurately identified while specificity describes the proportion of no/mild toxicity samples identified as such. This 3- or 5-fold cross-validation scheme was repeated 100 times in order to mitigate the effects of overfitting due to limited sample numbers. By repeating the cross-validation procedure and reporting the average results, it is possible to ensure that reported statistics are not overestimated due to how samples are assigned to training versus testing groups.

Functional analysis of antigen targets of toxicity-associated antibodies. The HOMER (v4.9) enrichment analysis tool and functional annotations from WikiPathways (www.wikipathways.org/) were used to determine the potential significance of the antigen targets of antibodies that were strongly differentially expressed between no/mild and severe toxicity groups.

### Discussion

It will be apparent from the foregoing description that immune-related toxicities are a significant barrier limiting the utility of ICI in melanoma treatment, particularly when given in combination [20]. Prior to the present disclosure, there was no predictive biomarker to identify patients who were likely to develop severe irAEs that can necessitate systemic immunosuppression or treatment termination. We analyzed whether a subset of metastatic melanoma patients possesses a sub-clinical autoimmune phenotype, characterized by a specific serum autoantibody profile, which predisposes them to develop severe irAEs following ICI therapy, in part due to enhanced recognition of self-antigens by T-cells. We used an unbiased proteomic microarray approach to compare global antibody levels in pre-treatment sera from melanoma patients treated with anti-CTLA-4, anti-PD-1, or the combination, and identified sets of toxicity-associated antibodies for each of the three treatment cohorts. Interestingly, the toxicity-associated antibody signatures were treatment-specific, with very little overlap across therapy groups, which without intending to be bound by any particular theory could be explained by the distinct cellular mechanisms of action for these treatments [21]. We found that the antigen targets for toxicity associated autoantibodies were significantly enriched for proteins that are highly expressed in organs affected by irAEs, and/or involved in cellular pathways that have been associated with immune pathology, suggesting a potential causative role for specific autoantibodies in development of irAEs. Finally, we generated SVM classifier models to identify sets of features (antibodies) that could be used to predict toxicity from baseline sera with excellent accuracy, sensitivity and specificity, demonstrating the potential utility of this approach to develop biomarker assays to guide the clinical management of melanoma patients treated with ICI.

By reinvigorating the immune system, immunomodulatory antibodies (anti-CTLA-4, anti PD-1) can promote anti-tumor immunity but also the development of irAEs. The precise mechanisms underlying irAEs induced by ICI are still unclear. Gastrointestinal (GI) irAEs have been associated with increased levels of neutrophil activation markers CD177 and CEACAM1, which are correlated with neutrophilic inflammation [10]. Additionally, it has been suggested that high baseline serum levels of IL-17, a cytokine that activates neutrophils, are associated with development of colitis following anti-CTLA-4 treatment [12]. A recent report also suggested that hypophysitis following anti-CTLA-4 treatment might be associated with development of antibodies, which were negative at baseline, against thyrotropin-, follicle-stimulating hormone-, and corticotropin-secreting pituitary gland cells [22]. While these pituitary-specific antibodies might mediate this irAE, they cannot be utilized as a predictive biomarker of treatment-induced pituitary toxicity as they are not detectable in pre-treatment sera. Other studies have failed to identify baseline serum antibodies associated with development of irAEs in immunotherapy-treated patients, although these focused solely on antibodies previously implicated in autoimmune diseases, such as anti-nuclear [23] or anti-thyroid [24] antibodies. ANAs, targeting both nuclear proteins and nucleic acid derivatives, comprise a large proportion of autoimmune disease-associated antibodies and have the most-recognized diagnostic and/or prognostic value for autoimmune diseases such as systemic lupus erythematosus (SLE) [25].

The present disclosure identifies significant enrichment of the protein targets of toxicity associated antibodies among functional pathways that have been associated with autoimmunity, such as TNF-α signaling [26], lung fibrosis [27], IL-1 [28] and TLR [29] signaling, and E. coli infection [30]. Interestingly, the present results also showed that the most differentially-expressed antibodies between no/mild and severe toxicity groups for each therapy group target protein antigens that are highly expressed in tissues affected by irAEs, including liver, skin, thyroid, pancreas, and adrenal gland [31]. In this regard, and without intending to be bound by theory, it is considered that specific baseline antibodies could predict the development of severe site-specific toxicities that are more clinically-significant; for example, severe hepatotoxicity versus severe skin toxicity. While the precise roles of toxicity-associated antibodies identified herein in mediating irAEs are yet to be established, their potential biological significance supports the present approach that involves the concept that a subset of antibodies promote the development of irAEs in patients treated with ICI. In view of the underlying similarities between the clinical manifestation of autoimmune disorders and irAEs induced by ICI, the presently provided data support a model in which some ICI-treated melanoma patients possess an underlying, subclinical autoimmune phenotype, which renders them susceptible to severe irAE development and is characterized by a specific baseline serum antibody profile. This autoimmune phenotype is likely to result from a combination of host- (germline), environment-, and tumor-specific factors.

While the present invention has been described through various specific embodiments, routine modification to these embodiments will be apparent to those skilled in the art, which modifications are intended to be included within the scope of this disclosure.

The following reference listing is not an indication that any particular reference is material to patentability.

### REFERENCES

This listing reference is not an indication that any particular reference is material to patentability.
1. Franklin C, Livingstone E, Roesch A, Schilling B, Schadendorf D. Immunotherapy in melanoma: Recent advances and future directions. European journal of surgical oncology: the journal of the European Society of Surgical Oncology and the British Association of Surgical Oncology 2017; 43(3):604-11 doi 10.1016/j.ejso.2016.07.145.
2. Hodi F S, Chesney J, Pavlick A C, Robert C, Grossmann K F, McDermott D F, et al. Combined nivolumab and ipilimumab versus ipilimumab alone in patients with advanced melanoma: 2-year overall survival outcomes in a multicentre, randomised, controlled, phase 2 trial. The Lancet Oncology 2016; 17(11):1558-68 doi 10.1016/s1470-2045(16)30366-7.
3. Day D, Hansen A R. Immune-Related Adverse Events Associated with Immune Checkpoint Inhibitors. BioDrugs: clinical immunotherapeutics, biopharmaceuticals and gene therapy 2016; 30(6):571-84 doi 10.1007/s40259016-0204-3.
4. Larkin J, Chiarion-Sileni V, Gonzalez R, Grob J J, Cowey C L, Lao C D, et al. Combined Nivolumab and Ipilimumab or Monotherapy in Untreated Melanoma. The New England journal of medicine 2015; 373(1):23-34 doi 10.1056/NEJMoa1504030.
5. Linardou H, Gogas H. Toxicity management of immunotherapy for patients with metastatic melanoma. Annals of translational medicine 2016; 4(14):272 doi 10.21037/atm.2016.07.10.
6. Eggermont A M, Chiarion-Sileni V, Grob J J, Dummer R, Wolchok J D, Schmidt H, et al. Prolonged Survival in Stage III Melanoma with Ipilimumab Adjuvant Therapy. The New England journal of medicine 2016; 375(19):1845-55 doi 10.1056/NEJMoa1611299.
7. Weber J, Mandala M, Del Vecchio M, Gogas H J, Arance A M, Cowey C L, et al. Adjuvant Nivolumab versus Ipilimumab in Resected Stage III or IV Melanoma. The New England journal of medicine 2017 doi 10.1056/NEJMoa1709030.
8. Gibney G T, Weiner L M, Atkins M B. Predictive biomarkers for checkpoint inhibitor-based immunotherapy. The Lancet Oncology 2016; 17(12):e542-e51 doi 10.1016/s1470-2045(16)30406-5.
9. Jacquelot N, Roberti M P, Enot D P, Rusakiewicz S, Ternes N, Jegou S, et al. Predictors of responses to immune checkpoint blockade in advanced melanoma. Nature communications 2017; 8(1):592 doi 10.1038/s41467-017-00608-2.
10. Shahabi V, Berman D, Chasalow S D, Wang L, Tsuchihashi Z, Hu B, et al. Gene expression profiling of whole blood in ipilimumab-treated patients for identification of potential biomarkers of immune-related gastrointestinal adverse events. Journal of translational medicine 2013; 11:75 doi 10.1186/1479-5876-11-75.
11. Subudhi S K, Aparicio A, Gao J, Zurita A J, Araujo J C, Logothetis C J, et al. Clonal expansion of CD8 T cells in the systemic circulation precedes development of ipilimumab-induced toxicities. Proceedings of the National Academy of Sciences of the United States of America 2016; 113(42):11919-24 doi 10.1073/pnas.1611421113.
12. Tarhini A A, Zahoor H, Lin Y, Malhotra U, Sander C, Butterfield L H, et al. Baseline circulating IL-17 predicts toxicity while TGF-beta1 and IL-10 are prognostic of relapse in ipilimumab neoadjuvant therapy of melanoma. Journal for immunotherapy of cancer 2015; 3:39 doi 10.1186/s40425-015-0081-1.
13. Champiat S, Lambotte O, Barreau E, Belkhir R, Berdelou A, Carbonnel F, et al. Management of immune checkpoint blockade dysimmune toxicities: a collaborative position paper. Annals of oncology: official journal of the European Society for Medical Oncology 2016; 27(4):559-74 doi 10.1093/annonc/mdv623.
14. Manson G, Norwood J, Marabelle A, Kohrt H, Houot R. Biomarkers associated with checkpoint inhibitors. Annals of oncology: official journal of the European Society for Medical Oncology 2016; 27(7):1199-206 doi 10.1093/annonc/mdw181.
15. Dubin K, Callahan M K, Ren B, Khanin R, Viale A, Ling L, et al. Intestinal microbiome analyses identify melanoma patients at risk for checkpoint-blockade-Induced colitis. Nature communications 2016; 7:10391 doi 10.1038/ncomms10391.
16. Kong Y C, Flynn J C. Opportunistic Autoimmune Disorders Potentiated by Immune-Checkpoint Inhibitors Anti-CTLA-4 and Anti-PD-1. Frontiers in immunology 2014; 5:206 doi 10.3389/fimmu.2014.00206.
17. Wich L G, Hamilton H K, Shapiro R L, Pavlick A, Berman R S, Polsky D, et al. Developing a multidisciplinary prospective melanoma biospecimen repository to advance translational research. American journal of translational research 2009; 1(1):35-43.
18. Jeong J S, Jiang L, Albino E, Marrero J, Rho H S, Hu J, et al. Rapid identification of monospecific monoclonal antibodies using a human proteome microarray. Molecular & cellular proteomics: MCP 2012; 11(6): O111 016253 doi 10.1074/mcp. 0111.016253.
19. Oken M M, Creech R H, Tormey D C, Horton J, Davis T E, McFadden E T, et al. Toxicity and response criteria of the Eastern Cooperative Oncology Group. American journal of clinical oncology 1982; 5(6):649-55.
20. Cousin S, Italiano A. Molecular Pathways: Immune Checkpoint Antibodies and their Toxicities. Clinical cancer research: an official journal of the American Association for Cancer Research 2016; 22(18):4550-5 doi 10.1158/10780432.ccr-15-2569.
21. Wei S C, Levine J H, Cogdill A P, Zhao Y, Anang N A S, Andrews M C, et al. Distinct Cellular Mechanisms Underlie Anti-CTLA-4 and Anti-PD-1 Checkpoint Blockade. Cell 2017 doi 10.1016/j.cell.2017.07.024.
22. Iwama S, De Remigis A, Callahan M K, Slovin S F, Wolchok J D, Caturegli P. Pituitary expression of CTLA-4 mediates hypophysitis secondary to administration of CTLA-4 blocking antibody. Science translational medicine 2014; 6(230):230ra45 doi 10.1126/scitranslmed.3008002.
23. Johncilla M, Misdraji J, Pratt D S, Agoston A T, Lauwers G Y, Srivastava A, et al. Ipilimumab-associated Hepatitis: Clinicopathologic Characterization in a Series of 11 Cases. The American journal of surgical pathology 2015; 39(8):1075-84 doi 10.1097/pas.0000000000000453.
24. Orlov S, Salari F, Kashat L, Walfish P G. Induction of painless thyroiditis in patients receiving programmed death 1 receptor immunotherapy for metastatic malignancies. The Journal of clinical endocrinology and metabolism 2015; 100(5):1738-41 doi 10.1210/jc.2014-4560.
25. Segni M, Pucarelli I, Truglia S, Turriziani I, Serafinelli C, Conti F. High prevalence of antinuclear antibodies in children with thyroid autoimmunity. Journal of immunology research 2014; 2014:150239 doi 10.1155/2014/150239.
26. Faustman D L, Davis M. TNF Receptor 2 and Disease: Autoimmunity and Regenerative Medicine. Frontiers in immunology 2013; 4:478 doi 10.3389/fimmu.2013.00478.
27. Hoyne G F, Elliott H, Mutsaers S E, Prele C M. Idiopathic pulmonary fibrosis and a role for autoimmunity. Immunology and cell biology 2017; 95(7):577-83 doi 10.1038/icb.2017.22.
28. Jamilloux Y, Belot A, Magnotti F, Benezech S, Gerfaud-Valentin M, Bourdonnay E, et al. Geoepidemiology and Immunologic Features of Autoinflammatory Diseases: a Comprehensive Review. Clinical reviews in allergy & immunology 2017 doi 10.1007/s12016-017-8613-8.
29. Chen J Q, Szodoray P, Zeher M. Toll-Like Receptor Pathways in Autoimmune Diseases. Clinical reviews in allergy & immunology 2016; 50(1):1-17 doi 10.1007/s12016-015-8473-z.
30. Singh V K, Yamaki K, Abe T, Shinohara T. Molecular mimicry between uveitopathogenic site of retinal S-antigen and Escherichia coli protein: induction of experimental autoimmune uveitis and lymphocyte cross-reaction. Cellular immunology 1989; 122(1):262-73.
31. Boutros C, Tarhini A, Routier E, Lambotte O, Ladurie F L, Carbonnel F, et al. Safety profiles of anti-CTLA-4 and anti-PD-1 antibodies alone and in combination. Nature reviews Clinical oncology 2016; 13(8):473-86 doi 10.1038/nrclinonc.2016.58.
32. Skerka C, Zipfel P F. Complement factor H related proteins in immune diseases. Vaccine 2008; 26 Suppl 8:19-14.
33. Rendleman J, Vogelsang M, Bapodra A, Adaniel C, Silva I, Moogk D, et al. Genetic associations of the interleukin locus at 1q32.1 with clinical outcomes of cutaneous melanoma. Journal of medical genetics 2015; 52(4):231-9 doi 10.1136/jmedgenet-2014-102832.
34. Vogelsang M, Martinez C N, Rendleman J, Bapodra A, Malecek K, Romanchuk A, et al. The Expression Quantitative Trait Loci in Immune Pathways and their Effect on Cutaneous Melanoma Prognosis. Clinical cancer research: an official journal of the American Association for Cancer Research 2016; 22(13):3268-80 doi 10.1158/1078-0432.ccr-15-2066.
35. Hakenberg O W. Nivolumab for the treatment of bladder cancer. Expert opinion on biological therapy 2017; 17(10):1309-15 doi 10.1080/14712598.2017.1353076.
36. Kreamer K M. Immune Checkpoint Blockade: A New Paradigm in Treating Advanced Cancer. Journal of the advanced practitioner in oncology 2014; 5(6):418-31.

What is claimed is:

1. A method comprising determining a signal from a composition of matter comprising detectably labeled antibodies and a first biological sample obtained from an individual who has cancer and who was not treated with a checkpoint inhibitor prior to obtaining the first biological sample, and a plurality of proteins attached to a substrate, the plurality of proteins selected from the proteins of Table 1, wherein at least some autoantibodies present in the first biological sample are bound to at least some of the proteins in the plurality of proteins, and wherein the signal is from the detectably labeled antibodies bound to the autoantibodies.

2. The method of claim 1, comprising comparing the signal to a reference to determine if the biological sample contained autoantibodies from an individual who:
i) is likely to exhibit no or mild toxicity from being treated with one or more checkpoint inhibitors; or
ii) is likely to exhibit severe toxicity from being treated with one or more checkpoint inhibitors;
the method optionally further comprising determining if the individual is likely exhibit to no, mild or severe toxicity to:
iii) treatment with a single checkpoint inhibitor that is an anti-Programmed cell death protein 1 (anti-PD-1) checkpoint inhibitor, and/or
vi) treatment with a single checkpoint inhibitor that is an anti-Cytotoxic T-lymphocyte-associated protein 4 (anti-CTLA-4) checkpoint inhibitor, and/or
v) treatment with a combination of an anti-PD-1 checkpoint inhibitor and an anti-CTLA-4 checkpoint inhibitor.

3. A method comprising treating an individual who has cancer with a checkpoint inhibitor, wherein:
a) if the individual is likely to exhibit no or mild toxicity according to claim 7, treating the individual with at least one checkpoint inhibitor without an agent that is used to reduce the risk of toxicity from treatment with the checkpoint inhibitor; or
b) if the individual is likely to exhibit severe toxicity according to claim 7, administering to the individual, prior to treatment with at least one checkpoint inhibitor, an agent to reduce the risk of toxicity, and concurrently or subsequently treating the individual with at least one checkpoint inhibitor.

4. The method of claim 3, comprising administering at least one checkpoint inhibitor to the individual without the agent that is used to reduce the risk of the toxicity.

5. The method of claim 3, wherein the individual is treated with only one checkpoint inhibitor that is an anti-PD-1 checkpoint inhibitor.

6. The method of claim 3, wherein the individual is treated with only one checkpoint inhibitor is an anti-CTLA-4 checkpoint inhibitor.

7. The method of claim 3, comprising treating the individual with a combination of the anti-PD-1 and the anti-CTLA-4 checkpoint inhibitors.

8. The method of claim 3, comprising administering the agent to reduce the risk of toxicity to the individual, and concurrently or subsequently treating the individual with at least one checkpoint inhibitor.

9. The method of claim 3, comprising testing a second biological sample from the individual during treating the individual with the one or more checkpoint inhibitors to monitor development of toxicity from the treatment.

10. The method of claim 3, wherein the individual has melanoma.
